(19) United States
(12) Patent Application Publication
(10) Pub. No.: US 2004/0210050 A1
(43) Pub. Date: Oct. 21, 2004

(54) Process for the preparation of highly pure cefuroxime axetil

(76) Inventors: Claudio Felisi, Zelo Buonpersico (IT); Davide Longoni, Gorgonzola (IT); Marco Alpegiani, Milano (IT); Walter Cabri, Rozanno (IT)

Correspondence Address: ROTHWELL, FIGG, ERNST & MANBECK, P.C., 1425 K STREET, N.W., SUITE 800, WASHINGTON, DC, 20005, US

(21) Appl. No.: 10/486,098

(22) PCT Filed: Aug. 1, 2002
(86) PCT No.: PCT/EP02/08583

(30) Foreign Application Priority Data
Aug. 10, 2001 (IT) MI2001A001763

(51) Int. Cl. C07D 501/14

(52) U.S. Cl. 540/228

(57) ABSTRACT

A process for the preparation of highly pure cefuroxime axetil is herein described. The process makes use of a treatment, which allows removing an impurity present in the reagent 1-acetoxyethyl bromide and responsible for the formation of cefuroxime dimeric derivatives. The removal of said impurity makes it easier to recover crystalline, cefuroxime axetil and allows obtaining an exceptional-quality product.

The present invention relates to a process for the preparation of highly pure cefuroxime axetil.

Cefuroxime axetil is the 1-acetoxyethyl ester of cefuroxime, a second-generation semisynthetic cephalosporin characterized by a broad spectrum activity against Gram-positive and Gram-negative bacteria. It is orally active and is marketed in the amorphous form, having this physical state better pharmacokinetic/pharmacodynamic characteristics than the crystalline product.

The conventional process for the preparation of cefuroxime axetil (Formula I) is the esterification of cefuroxime with 1-acetoxyethyl bromide (1-bromoethyl acetate), as disclosed in U.S. Pat. No. 4,267,320, to afford, in normal conditions, a crystalline product. The latter is transformed into the amorphous form using special techniques, as described, for example in U.S. Pat. Nos. 4,562,181; 4,820,833; 4,994,467 and 5,103,833.

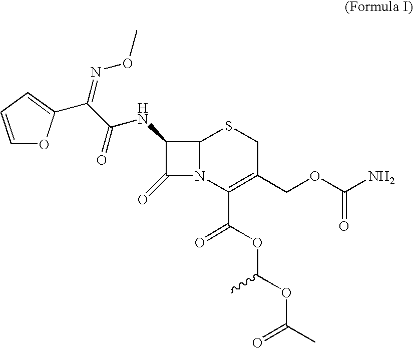

The preferred method for the preparation of amorphous cefuroxime axetil makes use of the spray drying technique. In these conditions, the quality of the amorphous product is directly related to that of the crystalline precursor, whose quality is therefore, in terms of purity and titre, of paramount importance.

The reagent used in the synthesis of 1-acetoxyethyl bromide has been found to be contaminated with different amounts of bis(1-bromoethyl)ether of formula (II).

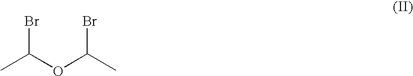

The amount of bis(1-bromoethyl)ether present in 1-acetoxyethyl bromide increases with storage and the formation and the increase in time occur independently of the synthesis method.

The presence of bis(1-bromoethyl)ether was demonstrated by analytical techniques (e.g., spectroscopy or chromatography) and comparison with literature data [Tetrahedron Letters, 29, 6489 (1988)].

Compound II reacts with cefuroxime to form dimeric impurities of formula (II) according to the following scheme:

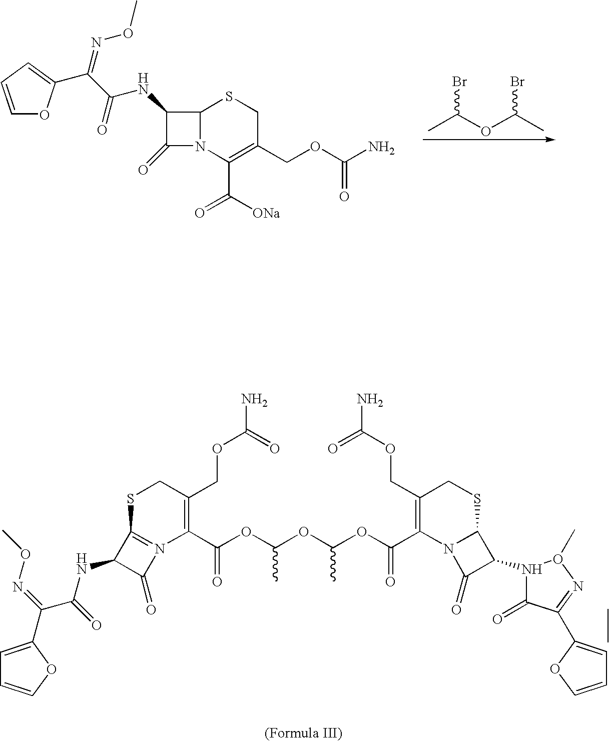

The reaction of cefuroxime with bis(1-bromoethyl)ether theoretically affords four diastereomers: the four of them have been spectroscopically detected and identified.

The presence of said dimeric derivatives of formula (III) makes the crystallization of cefuroxime axetil difficult and, above all, alters the quality of the resulting crystalline cefuroxime axetil. The conversion process of the crystalline product (by means of spray drying, freeze drying, roller drying techniques or solvent precipitation) into the amorphous one, i.e. the marketed form, does not improve the quality. It is therefore of utmost importance to obtain crystalline cefuroxime axetil having the highest quality.

It has now been found that bis(1-bromoethyl)ether can be removed and its formation can be prevented by treatment of crude 1-acetoxyethyl bromide with derivatives of formula (IV)

(R—COO)ₙM (IV)

wherein n is 1 or 2,

M is an alkali, alkaline-earth metal or ammonium,

R is hydrogen, alkyl or aryl optionally substituted with one more substituents selected from C₁-C₆ alkyl, phenyl, halogen, hydroxy, mercapto, amino, C₁-C₆ alkylthio, C₁-C₆ alkylamino, carboxy, —(CO₂)ₙM, —(SO₃)ₙM,

or R is a carboxy group optionally salified with M as counterion.

A particularly preferred compound of formula (IV) is sodium 2-ethyl hexanoate.

The treatment of crude 1-acetoxyethyl bromide with derivatives of formula (IV) can be carried out either on the liquid product as such or on the product dissolved in suitable organic solvents. Examples of suitable organic solvents comprise halogenated hydrocarbons (e.g., dichloromethane), carboxylic acid esters (e.g., ethyl acetate), ethers (e.g. tert-butyl methyl ether, tetrahydrofuran), carboxylic acid amides (e.g. N,N-dimethylacetamide, N-methylpyrrolidone), ketones (e.g., methyl ethyl ketone), dimethylcarbonate, sulfolane.

The treatment can be carried out at temperatures ranging from −20° C. to +40° C., for times ranging from a few minutes to some days or even longer.

The amount of derivative of formula (IV) to be used is evaluated on the basis of the amount of bis(1-bromoethyl)ether present in 1-acetoxyethyl bromide. Said amount can be calculated by means of conventional analytic techniques or tests. Typically, this amount ranges from some parts per thousand to some parts percent by weight compared with 1-acetoxyethyl bromide.

The following examples illustrate the invention in greater detail.

### COMPARATIVE EXAMPLE

146 ml of methylene chloride, 87.5 g (0.704 moles) of acetyl bromide and 0.15 g (0.0011 moles) of zinc chloride are placed at room temperature and under anhydrous atmosphere in a round-bottom flask.

The reaction mixture is cooled to 0+2° C., and 31.5 g (0.232 moles) of paraldehyde are added under stirring in about 45 minutes, keeping the reaction temperature below 5° C.

The reaction mixture is stirred for 1 hour, then washed with 146 ml of water pre-cooled at 5° C. After removing the aqueous layer, the organic phase is washed again twice, then concentrated under vacuum, keeping the bath temperature below 25° C.

The residue thus obtained is purified by distillation under vacuum.

About 100 g of 1-acetoxyethyl bromide in the form of colourless liquid with purity>90% (GC) are obtained. Yield 78%.

An aliquot of the resulting product (12;5 g) is used in the synthesis of cefuroxime axetil as reported in preparation n. 1 of U.S. Pat. No. 5,013,833.

18.8 g of cefuroxime axetil are obtained, wherein the total amount of the species corresponding to formula (II) is 2% (as determined by HPLC).

### EXAMPLE 1

146 ml of methylene chloride, 87.5 g (0.704 moles) of acetyl bromide and 0.15 g (0.0011 moles) of zinc chloride are added at room temperature in a round-bottom flask under anhydrous atmosphere.

The reaction mixture is cooled to 0+2° C. and 31.5 g (0.232 moles) of paraldehyde are added under stirring in about 45 minutes, keeping the reaction temperature below 5° C.

The reaction mixture is stirred for 1 hour, then washed with 146 ml of water at 5° C. and the resulting phases are separated.

The organic one is washed again twice, then concentrated under vacuum keeping the bath temperature below 25° C.

The residue thus obtained is purified by distillation under vacuum.

About 100 g of 1-acetoxyethyl bromide in the form of colourless liquid with purity>90% (GC) are obtained. Yield 78%.

The product is diluted with 100 g of N,N-dimethylacetamide at room temperature and 3 g (0.018 moles) of sodium 2-ethyl hexanoate are added to the solution, which is left to stand at 0° C. for 24 hours before use.

An aliquot of the solution (25 g) is used in the synthesis of cefuroxime axetil as reported in preparation n. 1 of U.S. Pat. No. 5,013,833.

19.2 g of cefuroxime axetil are obtained, wherein the species corresponding to formula (II) are absent (as determined by HPLC).

1. A process for the preparation of cefuroxime axetil by reaction of cefuroxime with 1-acetoxyethyl bromide, characterized in that 1-acetoxyethyl bromide is previously treated with a compound of formula (IV)
(R—COO)ₙM (IV)
wherein
n is 1 and 2,
M is an alkali, alkaline-earth metal or ammonium,
R is hydrogen, alkyl or aryl, optionally substituted with one or more substituents selected from C₁-C₆ alkyl, phenyl, halogen, hydroxy, mercapto, amino, C₁-C₆ alkylthio, C₁-C₆ alkylamino, carboxy,
or is a group of formula —(CO₂)ₙM, or —(SO₃)ₙM, wherein M and n are as defined above.

2. A process as claimed in claim 1 wherein the product of formula (IV) is sodium 2-ethyl hexanoate.

3. A process as claimed in claim 1 wherein the treatment is effected in N,N-dimethylacetamide.

4. Cefuroxime axetil substantially free from dimeric derivatives of formula (III).
